(19) United States
(12) Patent Application Publication
(10) Pub. No.: US 2009/0325183 A1
(43) Pub. Date: Dec. 31, 2009

(54) SEQUENCING METHODS

(75) Inventors: Kai Qin Lao, Pleasanton, CA (US); Neil A. Straus, Emeryville, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/553,928

(22) Filed: Sep. 3, 2009

Related U.S. Application Data
(63) Continuation of application No. 11/956,100, filed on Dec. 13, 2007, now abandoned.
(60) Provisional application No. 60/874,868, filed on Dec. 14, 2006.

(51) Int. Cl. C12Q 1/68

(52) U.S. Cl. 435/6

(57) ABSTRACT

The present teachings provide methods and compositions for sequencing one or more target nucleic acids. High levels of multiplexing are provided by the use of an emulsion PCR comprising primer-immobilized beads. The resulting reaction products can be sequenced by any of a variety of mobility-dependent analytical techniques, such as mass spectrometry. In some embodiments, a first collection of amplification products on a first collection of beads are transferred to a second collection of beads. In some embodiments, a first collection of amplification products on a first collection of beads is amplified in a rolling circle amplification reaction. The present teachings also provide compositions, kits, and devices for performing and sequencing the products of the emulsion amplification reactions as described herein.

### CROSS-REFERENCE TO RELATED APPLICATIONS

The present application is a Continuation of U.S. patent application Ser. No. 11/956,100, filed on Dec. 13, 2007, and claims priority to U.S. Provisional Application No. 60/874,868, filed Dec. 13, 2006, which is incorporated by reference in its entirety.

### FIELD

The present teachings are in the field of molecular and cell biology, specifically in the field of sequencing target nucleic acids, for example using emulsion amplification reactions and primer-encoded beads.

### BACKGROUND

Numerous fields in molecular biology require knowing the sequence of target nucleic acids. The increasing amount of sequence information available to scientists (see Venter et al., Science. 2001 Feb. 16; 291(5507):1304-51, and Adams et al., Science 2000 Mar. 24; 287(5461):2185-95) in the post-genomics era has produced an increased need for rapid, reliable, low-cost, high-throughput, sensitive, and accurate methods to sequence complex nucleic acid samples. One approach to sequencing nucleic acids is to use mobility-dependent analysis techniques, such as capillary electrophoresis (see for example U.S. Pat. No. 5,207,886; U.S. Pat. No. 5,240,576; U.S. Pat. No. 5,374,527; and U.S. Pat. No. 5,597,468) and MALDI-TOF mass spectrometry (Smith et al., Nature 14: 1084, 1996; Koster et al. Nature 14: 1123, 1996; Edwards et al., NAR 29:e104, 2001; U.S. Pat. No. 5,643,798; U.S. Pat. No. 5,288,644; and U.S. Pat. No. 5,453,247).

### SUMMARY

In some embodiments, the present teachings provide methods for determining the sequence a target nucleic acid. The target nucleic acid is amplified in an emulsion amplification reaction using a primer-encoded bead to form an extension product bead. The extension products are then subjected to a mobility-dependent analytical technique, typically a sequencing technique. In some embodiments the extension product bead is amplified prior to sequencing, for example, by transferring the bead to a micro-titer plate, adding additional primer-encoded beads and using amplification reactions to make multiple extension product beads. Sequencing may comprise, for example, performing chain terminating reactions on the extension product bead or beads and subsequently analyzing the resulting mixed-length reaction products. In some embodiments analysis may be by mass spectrometry or capillary electrophoresis.

In some embodiments, the present teachings provide methods of sequencing a target nucleic acid comprising; amplifying the target nucleic acid in an emulsion amplification reaction, wherein the emulsion amplification reaction comprises a primer-encoded bead, to form an extension product bead; performing a chain-terminating reaction on the extension-product bead to form a plurality of mixed-length extension products; eluting the mixed-length extension products; and, determining the masses of the mixed-length products to sequence the target nucleic acid.

Additional methods and compositions, as well as kits, are also provided.

### BRIEF DESCRIPTION OF THE DRAWINGS

The skilled artisan will understand that the drawings, described below, are for illustration purposes only. The drawings are not intended to limit the scope of the present teachings in any way.

FIG. 1 depicts one workflow according to some embodiments of the present teachings.

FIG. 2 depicts certain aspects of various compositions and methods according to some embodiments of the present teachings.

FIG. 3 depicts certain aspects of various compositions and methods according to some embodiments of the present teachings.

FIG. 4 depicts certain aspects of various compositions and methods according to some embodiments of the present teachings.

### DESCRIPTION OF EXEMPLARY EMBODIMENTS

Aspects of the present teachings may be further understood in light of the following examples, which should not be construed as limiting the scope of the present teachings in any way. The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

The practice of the disclosed methods and compositions may employ, unless otherwise indicated, conventional techniques and descriptions of organic chemistry, polymer technology, molecular biology (including recombinant techniques), cell biology, biochemistry, and immunology, which are within the skill of the art. Such conventional techniques include oligonucleotide synthesis, and hybridization, extension reactions, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. The same holds true for ranges in increments of 10⁵, 10⁴, 10³, 10², 10, 10⁻¹, 10⁻², 10⁻³, 10⁻⁴, or 10⁻⁵, for example. This applies regardless of the breadth of the range.

### Some Definitions

As used herein, the term “target nucleic acid” refers to a polynucleotide sequence that is sought to be amplified and sequenced. The target nucleic can be obtained from any source, and can comprise any number of different compositional components. For example, the target nucleic acid can be DNA, RNA, transfer RNA, siRNA, and can comprise nucleic acid analogs or other nucleic acid mimics. In some embodiments the target nucleic acids will be fragmented genomic DNA (gDNA), micro RNAs (miRNAs) or other short RNAs. The target can be methylated, non-methylated, or both. The target can be bisulfite-treated and have non-methylated cytosines converted to uracil. Further, it will be appreciated that “target nucleic acid” can refer to the target nucleic acid itself, as well as surrogates thereof, for example amplification products, and native sequences. In some embodiments, a short target nucleic acid is a short DNA molecule derived from a degraded source, such as can be found in for example but not limited to forensics samples (see for example Butler, 2001, Forensic DNA Typing: Biology and Technology Behind STR Markers.

The target nucleic acid of the present teachings can be derived from any of a number of sources, including without limitation, viruses, prokaryotes, eukaryotes, for example but not limited to plants, fungi, and animals. These sources may include, but are not limited to, whole blood, a tissue biopsy, lymph, bone marrow, amniotic fluid, hair, skin, semen, biowarfare agents, anal secretions, vaginal secretions, perspiration, saliva, buccal swabs, various environmental samples (for example, agricultural, water, and soil), research samples generally, purified samples generally, cultured cells, and lysed cells. It will be appreciated that target nucleic acids can be isolated from samples using any of a variety of procedures known in the art, for example the Applied Biosystems ABI Prism™ 6100 Nucleic Acid PrepStation, and the ABI Prism™ 6700 Automated Nucleic Acid Workstation, Boom et al., U.S. Pat. No. 5,234,809, mirVana RNA isolation kit (Ambion), etc. It will be appreciated that polynucleotides can be cut or sheared prior to analysis, including the use of such procedures as mechanical force, sonication, restriction endonuclease cleavage, or any method known in the art, to produce target nucleic acids.

In general, the target nucleic acids of the present teachings will be single stranded, though in some embodiments the target nucleic acids can be double stranded, and/or comprise double-stranded regions due to secondary structure, and a single strand can result from denaturation.

As used herein, the term “hybridization” refers to the complementary base-pairing interaction of one nucleic acid with another nucleic acid that results in the formation of a duplex, triplex, or other higher-ordered structure, and is used herein interchangeably with “annealing.” Typically, the primary interaction is base specific, e.g., A/T and G/C, by Watson/Crick and Hoogsteen-type hydrogen bonding. Base-stacking and hydrophobic interactions can also contribute to duplex stability. Conditions for hybridizing primers to complementary and substantially complementary target sequences are well known, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the polynucleotides and the complementary region, the pH, the temperature, the presence of mono- and divalent cations, the proportion of G and C nucleotides in the hybridizing region, the viscosity of the medium, and the presence of denaturants. Such variables also influence the time required for hybridization. Thus, the preferred annealing conditions will depend upon the particular application. Such conditions, however, can be routinely determined by the person of ordinary skill in the art without undue experimentation. It will be appreciated that complementarity need not be perfect; there can be a small number of base pair mismatches that will minimally interfere with hybridization between the target sequence and the primers of the present teachings. However, if the number of base pair mismatches is so great that no hybridization can occur under minimally stringent conditions then the sequence is generally not a complementary target sequence.

As used herein, the term “mobility-dependent analytical technique” as used herein refers to any means for separating different molecular species based on differential rates of migration of those different molecular species in one or more separation techniques. Exemplary mobility-dependent analysis techniques include gel electrophoresis, capillary electrophoresis, chromatography, capillary electrochromatography, mass spectroscopy, sedimentation, e.g., gradient centrifugation, field-flow fractionation, multi-stage extraction techniques and the like. Descriptions of mobility-dependent analytical techniques can be found in, among other places, U.S. Pat. Nos. 5,470,705, 5,514,543, 5,580,732, 5,624,800, and 5,807,682, PCT Publication No. WO 01/92579, Fu et al., Current Opinion in Biotechnology, 2003, 14:1:96-100, D. R. Baker, Capillary Electrophoresis, Wiley-Interscience (1995), Biochromatography: Theory and Practice, M. A. Vijayalakshmi, ed., Taylor & Francis, London, U.K. (2003); and A. Pingoud et al., Biochemical Methods: A Concise Guide for Students and Researchers, Wiley-VCH Verlag GmbH, Weinheim, Germany (2002).

### Exemplary Embodiments

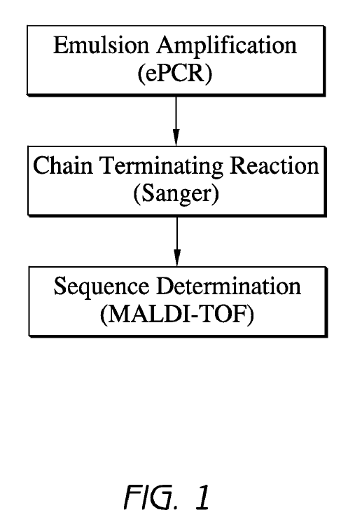

FIG. 1 depicts a workflow according to some embodiments of the present teachings. Here, an emulsion amplification reaction, such as for example an emulsion PCR (ePCR), is performed on one or more target nucleic acids. The resulting amplification products can undergo a chain-terminating reaction, for example a chain-terminating reaction with a dideoxynucleotide, such as a Sanger reaction. The resulting mixed-length extension products can be analyzed, for example by detection by mass spectrometry (e.g. MALDI-TOF), thus allowing for the sequence determination of the target nucleic acid.

One or more primers can be immobilized on a bead to form a primer-encoded bead. Typically a primer encoded bead comprises a plurality of immobilized copies of the same primer. As described below, an adapter that is complementary to the immobilized primer may be ligated to a target nucleic acid. In an extension reaction one or more of the immobilized primers on the bead can be extended in the presence of the target nucleic acid to form a first strand extension product. A bead comprising one or more such first strand products can be referred to as an extension-product bead. The target nucleic acid can also be modified with a second adapter such that a complementary primer can be used to make a second strand extension product, that is, a strand that corresponds to the target nucleic acid.

In some embodiments, methods of sequencing a target nucleic acid comprise; amplifying the target nucleic acid in an emulsion amplification reaction, wherein the emulsion amplification reaction comprises a primer-encoded bead, to form an extension product bead; performing a chain-terminating reaction on the extension-product bead to form a collection of mixed-length extension products; eluting the mixed-length extension products; and, determining the masses of the mixed-length products to sequence the target nucleic acid.

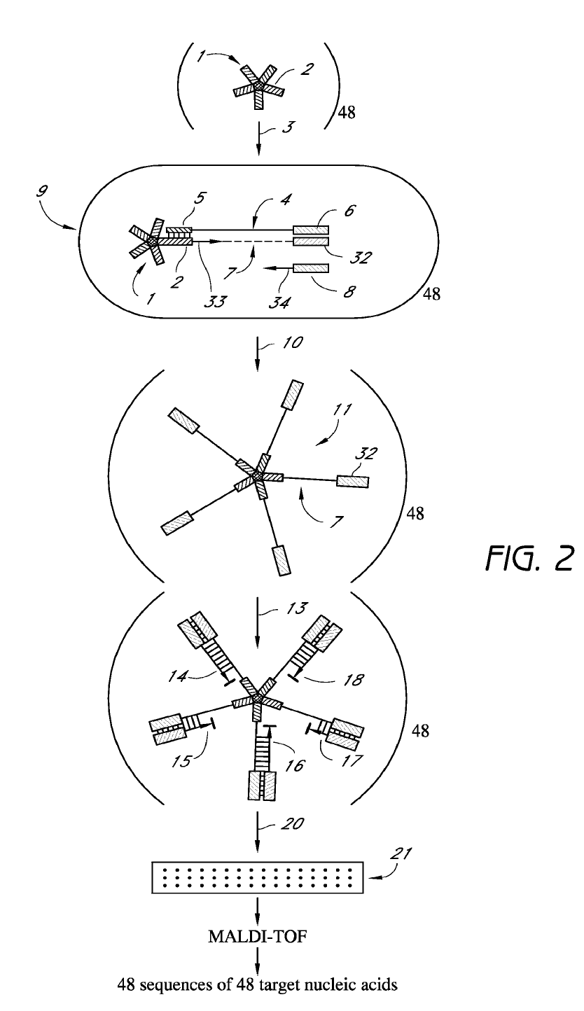

FIG. 2 depicts a process and some compositions according to the present teachings. Here, a primer-encoded bead (1), containing a plurality of immobilized primers (any one of which (2) is shown), can undergo a hybridization reaction (3) with a target nucleic acid (4). (The reaction can have a plurality of target nucleic acids, stoichiometrically set-up to allow for one molecule of target nucleic acid to interact with one bead in one aqueous droplet. For this illustration, 48 reactions on 48 different target nucleic acids are considered to be occurring separately.) The target nucleic acids can be prepared in such fashion (using conventional approaches such as restriction digestion and adapter ligation) so as to have a first end (5), which hybridizes to the primer (2) of the primer-encoded bead (1). The target nucleic acid (4) can further have a second end (6), which can serve as the sequence of a second primer (8) in a PCR. The first end (5) and second end (6) may be, for example, universal primer adaptors ligated to the ends of the target nucleic acid (4).

Specifically, the first primer from the bead (2) can hybridize to the first end (5) of the target nucleic acid (4), and be extended (33) to form a first strand extension product (7; dashed line). The resulting first strand extension product (7) thus contains a sequence (32) complementary to the second end of the target nucleic acid (6). The second primer (8) of the PCR can thus hybridize to the corresponding end sequence (32) and be extended (34) making a second strand product.

As a result of PCR cycling, the immobilized primers of the beads become extended; thus the bead bears a collection of first strand extension products complementary to the target nucleic acid. This reaction can occur in the context of a droplet (9) of a water-in oil-emulsion. As a result of the reaction (10), a bead (11) results that contains a collection of first strand extension products (7), all of which for a given bead correspond to a single target nucleic acid (4).

The first strand extension products (7) can then be sequenced. Hybridization (13) of primers complementary to the 3′ ends of the first strand products on the bead can be performed, and a chain-terminating reaction (e.g. Sanger) performed, resulting in a plurality of mixed-length reaction products (14, 15, 16, 17, and 18). Typically a collection of beads will be present in the emulsion from which drop (9), containing a single bead (1), is obtained, thus resulting in the generation of a collection of beads containing first strand extension products, and, following the chain-terminating reactions, a collection of beads containing a plurality of mixed-length reaction products. Because the stoichiometry of the emulsion PCR is performed in such fashion as to allow for a single target nucleic acid molecule to be present in each aqueous drop (9), eventually, each bead of the collection of beads containing a plurality of mixed-length reaction products represents a single target nucleic acid. Thus, for example, a collection of 48 beads can result in 48 beads each of which contains a plurality of mixed-length reaction products representing a single target nucleic acid. (Although illustrated with a collection of 48 beads, any number of beads may be used.) Dispersing (20) these 48 beads containing their mixed-length reaction products into a MALDI-Plate (21), can allow for mass spectrometry-based sequencing of each of the 48 target nucleic acids represented on the 48 beads. Other mobility dependent analytical methods, such as capillary electrophoresis, may be used.

Methods of sequencing target nucleic acids with MALDI-TOF can be found, for example, in Smith et al., Nature 14: 1084, 1996; Koster et al. Nature 14: 1123, 1996; Edwards et al., NAR 29:e104, 2001; U.S. Pat. No. 5,643,798; U.S. Pat. No. 5,288,644; and U.S. Pat. No. 5,453,247.

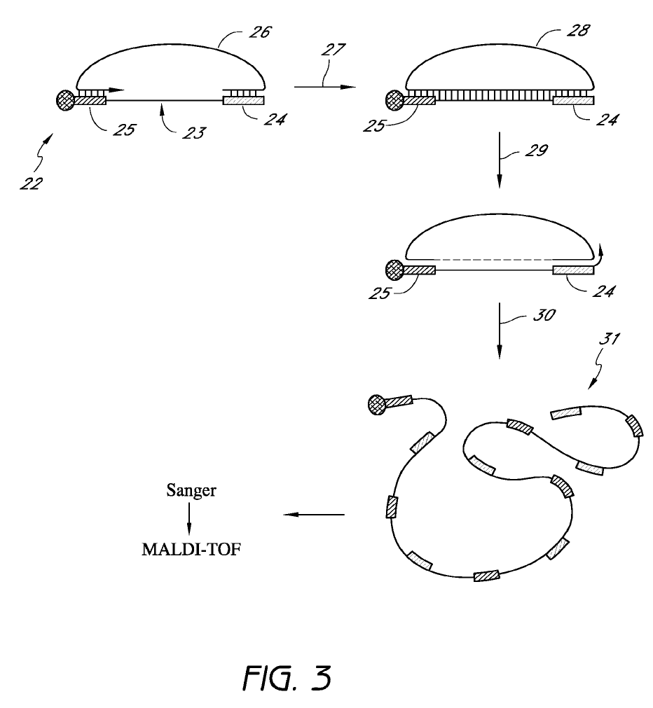

In some embodiments, it may be desirable to further amplify the first extension products on a bead. FIG. 3 depicts a rolling circle amplification reaction of one of the beads resulting from an e-PCR. For example, a first extension product (23) of a bead (22) contains a first end (25) and a second end (24), to which a nucleic acid probe (26) can be designed to hybridize. (Of course, the depicted bead (22) can further comprise additional first strand extension products, though for simplicity, here FIG. 3 only depicts one such first strand extension product (23)). The nucleic acid probe can be extended (arrow), and the two ends ligated together (27) to form a circle (28).

The resulting circle (28) can be amplified in a rolling circle amplification reaction (29). As shown, the second end (24, with arrow) of the extension product can be used as the primer in such a rolling circle amplification reaction. (In some embodiments, a separate primer molecule can be hybridized to the circle, and rolling circle amplification can proceed therefrom.) As a result (30), the beads now contain a concatameric plurality of rolling circle-amplified first extension products (31) emanating from the original first strand extension product. Thereafter, a chain-terminating Sanger reaction employing primers directed to one of the ends of the extension product will have a collection of sites on which to hybridize, thus increasing the number of mixed length extension products on a given bead. By increasing the number of mixed length extension products on a given bead, the sensitivity of mass spectrometry can be more easily met.

In some embodiments a transfer amplification process is used to amplify the number of first extension products that can be used for sequencing. For example, one bead comprising first extension products generated in an e-PCR (such as bead 11 in FIG. 2) can be transferred to a container, such as a well of a micro-titer plate. Additional primer-encoded beads and free primer complementary to the end of the extension product (such as primer 8 in FIG. 2) are added to the container and a PCR is used to generate first extension products on the additional beads. This provides another avenue to increase the number of molecules corresponding to a given target nucleic acid that can be used in mobility-dependent analytical techniques. For example, these methods can be used to more easily meet the sensitivity requirements of mass spectrometry. Of course, such a transfer PCR can be employed in any of a variety of contexts and methods. Thus, the additional amplification products can be analyzed using any of a variety of mobility dependant analysis techniques, including capillary electrophoresis. The transfer amplification process can be employed in any context in which a greater number of molecules are desired for analysis.

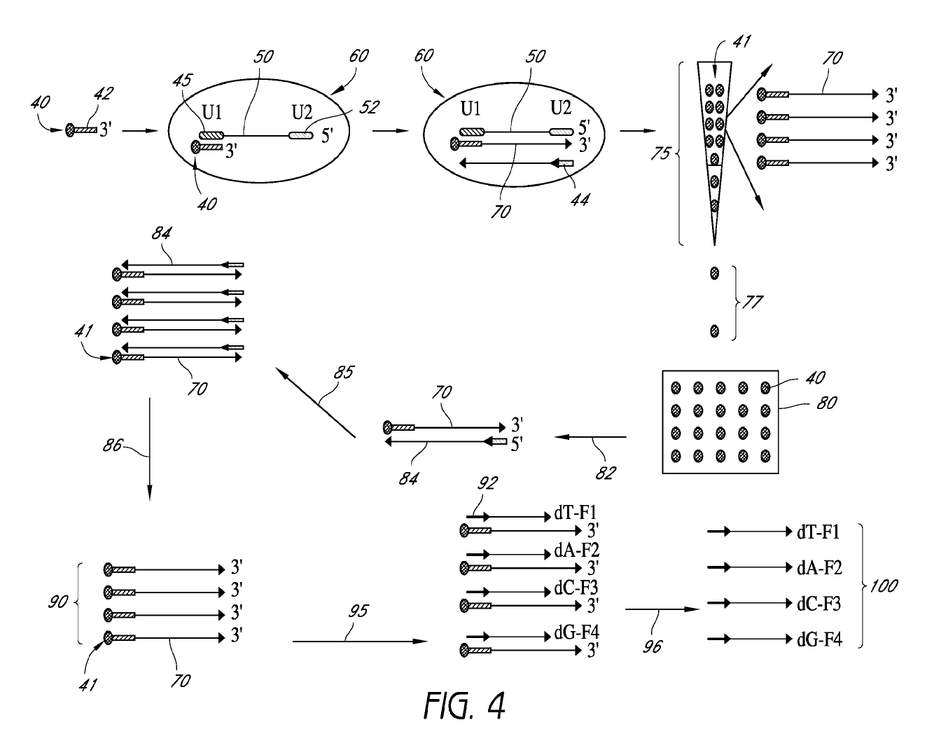

FIG. 4 depicts an exemplary transfer amplification process using PCR. Target nucleic acids are initially prepared for analysis. For example, gDNA may be fragmented by enzymatic cuts, sheer force or thermal heating to produce a collection of target nucleic acids to be analyzed. In this example fragments may be about 1 to 2 kb in length, although other lengths may be used. Primer adapters are then ligated to the 3′ and 5′ ends of the target nucleic acids, here the ends of the gDNA fragments. Beads (40) comprising a first primer (42), such as a first universal primer, are prepared. The first primer (42) is complementary to the 3′ adapter (45) on a target nucleic acid (or to a portion of the 3′ end of the target if adapters are not utilized, such as if the target sequence is known). Target nucleic acids (50) with the ligated 3′ (45) and 5′ (52) adapters are mixed with the beads (40) comprising the first primer (42). Oil is added and emulsion droplets (60) are formed comprising only one target nucleic acid (50) and one bead (40). A second primer (44) is added to each emulsion droplet (60), where the second primer (44) corresponds to the 5′ adapter (52) (or to a portion of the 5′ end of the target if adapters are not utilized, such as if the target sequence is known) and ePCR is performed in the emulsion droplets, resulting in the extension of the immobilized primers (42) on the bead (40) to produce first strand extension products (70).

Beads (41) (comprising first strand extension products (70)) from each emulsion droplet (60) are collected, oil and other reagents including unreacted primer are washed away and the beads (41) are separated, for example by use of a bead sorting instrument (75). Beads are then dispensed (77) into separate containers, such as individual wells of a micro-titer plate (80), one bead (40) to a well. The micro-titer plate may be, for example, a 96-well, 384-well, or 1536-well plate. Of course other size plates and types of plates may be selected by the skilled artisan, depending on the particular circumstances. For example, for MALDI-TOF sequencing nano-well plates may be used, which may comprise 250,000 wells.

Additional beads (40) comprising the first primer (42) are added (82) to the well along with free second primer (44). Alternatively, the additional primer-encoded beads (40) and free second primer (44) may be preloaded in the wells prior to dispensing the beads (41) comprising the first strand extension products (70). A PCR is run, resulting initially in a second strand extension product (84), which in turn allows for extension of the immobilized primers (42) on the additional beads (85) to produce first strand extension products (70) on each of the additional beads. In this way the first strand extension products are “transferred” to the new beads. Unreacted primers are washed away and second strands are removed (86), such that each well comprises a collection of beads (90), where each bead comprises one or more first strand extension products (70) that are complementary to a single target nucleic acid (50). Hybridization of primers (92) complementary to the 3′ ends of the first strand products (70) on the beads (41) can be performed and a chain terminating reaction (e.g. Sanger) performed (95) using dNTPs and ddNTPs (which may be labeled, for example, for capillary electrophoresis), resulting after purification (96) in a plurality of mixed length products (100). The mixed length products (100) can then be analyzed to determine the sequence of the target nucleic acid (50) in each well. For example, they may be injected into a capillary electrophoresis instrument. In other embodiments the beads comprising the mixed length products may be dispersed into a MALDI-Plate to allow for mass spectrometry based sequencing.

In some embodiments, e-PCR and chain-termination reactions can be followed by bead immobilization in a conventional microtitre plate, followed by elution of the mixed-length reaction products. The eluted mixed length reaction products can then be gridded on a MALDI-plate.

In some embodiments, the e-PCR and chain termination reactions can be followed directly by bead immobilization on a MALDI-plate. In one embodiment the beads are transferred into a gold plate to form a bead array, MALDI matrix is added to immobilize the beads and the mixed length products that were hybridized to the first strand extension products are released and analyzed.

In some embodiments, the ePCR and chain termination reactions are followed by fluorescent activated-cell sorting (FACS) into a MALDI-plate. Any of a variety of sorting procedures can be used. For illustrative teachings, see for example U.S. Patent Application US20040036870A1, U.S. Pat. No. 6,816,257, U.S. Pat. No. 6,710,871, and U.S. Patent Application US20020028434A1.

In some embodiments, the e-PCR can be followed by an enrichment procedure. For example, enrichment can be performed to preferentially select for those beads that were in an aqueous drop and contained a single molecule of target nucleic acid, and which now contain a collection of first stand extension products. For example, a fluorescent labeled nucleic acid complementary to a sequence in the first stand extension products can be employed, and those beads which light-up, and are sorted via FACS, are thus enriched for reaction products.

Methods of manipulating beads, and performing sequencing reactions in high density plates, are discussed for example in Nature, 2005, 437 (7057): 376-380.

Methods of using sequence tags along with MALDI-TOF-based sequencing can be found for example in EP 1 206 577 B1.

Methods of performing emulsion PCR can be found, for example, in Nature Methods, Vol 3, No. 7, July 2006.

Methods of performing emulsion PCR with beads, as well as methods of FACS-sorting such beads, and performing enrichment, can be found for example in Dressman et., PNAS, vol 100, no. 15, 8817-8822.

More generally, methods of sequencing target nucleic acids with MALDI-TOF can be found, for example, in Smith et al., Nature 14: 1084, 1996; Koster et al. Nature 14: 1123, 1996; Edwards et al., NAR 29:e104, 2001; U.S. Pat. No. 5,643,798; U.S. Pat. No. 5,288,644; and U.S. Pat. No. 5,453,247.

In addition, methods of sequencing target nucleic acids by capillary electrophoresis can be found, for example, in U.S. Pat. No. 5,207,886; U.S. Pat. No. 5,240,576; U.S. Pat. No. 5,374,527; and U.S. Pat. No. 5,597,468.

### Kits

In certain embodiments, the present teachings also provide kits designed to expedite performing certain methods. In some embodiments, kits serve to expedite the performance of the methods of interest by assembling two or more components used in carrying out the methods. In some embodiments, kits may contain components in pre-measured unit amounts to minimize the need for measurements by end-users. In some embodiments, kits may include instructions for performing one or more methods of the present teachings. In certain embodiments, the kit components are optimized to operate in conjunction with one another.

Thus, in some embodiments the present teachings provide a kit for sequencing comprising reagents for emulsion PCR, reagents for chain-terminating reactions, reagents for mobility-dependent analytical techniques, such as MALDI-TOF or capillary electrophoresis, and optionally reagents for performing a transfer PCR.

While the present teachings have been described in terms of these exemplary embodiments, the skilled artisan will readily understand that numerous variations and modifications of these exemplary embodiments are possible without undue experimentation. All such variations and modifications are within the scope of the current teachings. Aspects of the present teachings may be further understood in light of the following example, which should not be construed as limiting the scope of the teachings in any way.

Although the disclosed teachings have been described with reference to various applications, methods, kits, and compositions, it will be appreciated that various changes and modifications may be made without departing from the teachings herein and the claimed invention below. The foregoing examples are provided to better illustrate the disclosed teachings and are not intended to limit the scope of the teachings presented herein.

1. A method of sequencing a target nucleic acid comprising;
amplifying the target nucleic acid in an emulsion amplification reaction, wherein the emulsion amplification reaction comprises a primer-encoded bead, to form an extension product bead comprising a plurality of first strand extension products;
performing a chain-terminating reaction on the extension-product bead to form a plurality of mixed-length extension products;
eluting the mixed-length extension products; and,
determining the masses of the mixed-length products to sequence the target nucleic acid.

2. The method according to claim 1 wherein the plurality of first strand extension products on the bead are amplified in a transfer amplification reaction prior to the chain-terminating reaction.

3. The method according to claim 1 where the plurality of first strand extension products on the bead are amplified in a rolling circle amplification reaction prior to the chain-terminating reaction.

4. The method of claim 1 additionally comprising forming one or more additional extension-product beads in a transfer amplification process prior to determining the sequence of the extension products.
